(19) United States
(12) Patent Application Publication
(10) Pub. No.: US 2024/0335306 A1
(43) Pub. Date: Oct. 10, 2024

(54) LOW RADIAL FORCE LOCALIZING STENTS

(71) Applicant: BVW Holding AG, Cham (CN)

(72) Inventors: Lukas Bluecher, Eurasberg (DE); Michael Milbocker, Holliston, MA (US)

(21) Appl. No.: 18/621,242

(22) Filed: Mar. 29, 2024

Related U.S. Application Data
(60) Provisional application No. 63/456,838, filed on Apr. 4, 2023.

(51) Int. Cl. A61F 2/848; A61F 2/00

(52) U.S. Cl. CPC A61F 2/848; A61F 2002/0081; A61F 2210/0014; A61F 2220/0008

(57) ABSTRACT

Low radial force stents with good resistance to migration are described comprising microstructured surfaces which generate inward radially directed grip to a lumen. In particular, stents are described for deployment within biological lumens where a novel combination of low outwardly directed radial force and resistance to shear slippage within the lumen is achieved by hierarchical microstructured surfaces which provide non-frictional grip to the luminal surface. Combinations of microstructured surfaces which combine low radial force frictional grip and non-frictional grip which do not rely on axially dependent changes in stent diameter or stent oversizing are described. These combinations of microstructured surfaces when placed on the outer surface of a stent provide a non-migrating stent. The hierarchical levels of the disclosed microstructures may themselves by composites of microstructures, which may or may not be self-similar to other hierarchical levels.

### CROSS-REFERENCES TO RELATED APPLICATIONS

This application claims benefit of the following patent application(s) which is/are hereby incorporated by reference: U.S. Provisional App. No. 63/456,838 filed on Apr. 4, 2023.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

### Not Applicable

### REFERENCE TO SEQUENCE LISTING OR COMPUTER PROGRAM LISTING APPENDIX

Not Applicable

### BACKGROUND OF THE DISCLOSURE

Minimally invasive, percutaneous implantation techniques has found increased acceptance as alternatives to conventional surgery for cardiac patients at high risk of morbidity and mortality. Minimally invasive procedures have been developed for the treatment of nonvascular lumenal defects. Generally, minimally invasive implantation techniques may likely become the predominant means of treating many, if not all, lumenal diseases.

Percutaneous pulmonary valve implantation can be performed safely, especially in patients who have undergone surgery on the right ventricular outflow tract during repair of congenital heart disease. Nitinol stents are commonly used for minimally invasive delivery and anchoring of heart valve prostheses. The large elastic strains that nitinol withstands reduces the risk of stent damage during implantation, while enabling the large diameter reductions required for minimally invasive delivery.

Accurate sizing of the stent with respect to fixation in the host lumen should be considered because insufficient matching of the prosthetic to tissue can result in migration of the stent from the target location. More particularly, regarding cardiac valve repair, stent migration can result in a more serious condition known as paravalvular leakage. On the other hand, excessive radial fixation force on the tissue can be associated with the risk of tissue wall degeneration and damage.

Stent migration and lumenal wall damage remain primary concerns in all stent implantations. Unfortunately, amelioration of these two failure modes demands opposing design criteria. Stent migration can be reduced by increased stent radial force, while reduced luminal damage corresponds to reduced stent radial force.

In addition to wall degeneration and damage, matching the stent size to the size of the vessel is complicated by the continuous and radially outward directed expanding force that self-expandable stents exert after deployment leading to negative chronic recoil and a larger vessel after follow-up. Therefore, there is a need to reduce the radial force exerted by the stent not only for reducing wall degeneration and damage, but also for reducing chronic recoil and larger vessel size. Because excessive radial force in vascular stents is needed to provide a large positive radial force anchoring the stent, the negative effects of this positive radial force opposes the conditions for proper fixation and function.

Stent oversizing and excessive radial force can further lead to tissue remodeling. The presence of a permanent stent, and the amount of force it exerts, is known to affect the host tissue with a risk of triggering adverse biological processes such as thrombosis, in-stent restenosis, and neo intimal proliferation. Hence, there is a need for a stent localization function which does not require stent oversizing to produce the desired fixation result. It is understood by those skilled in the art that tissue remodeling problems are not limited to cardiac stents, and hence the stents of the present disclosure are useful in all medical stenting applications.

In-vivo functionality assessment related to stent localization and stent fit comprises a variety of mechanical parameters. Fortunately, the ratios of stent parameters fall within certain ranges across stent types if one decouples the anti-migration features from proper fit features. For the first time, the mechanical characteristics and structural features of a new class of low radial force stents is disclosed herein.

### BRIEF SUMMARY

The embodiments disclosed herein may be composed of a repeating design of four rings of 40 struts, connected by tilted bridges. In some embodiments, the internal stent diameter may be approximately 30 mm and the struts may have a cross sectional dimension of approximately 0.3 mm×0.4 mm on average.

Parallel plate compression (crush test) involves placing stents between parallel plates at room temperature on an Instron machine which applies pressure across the plates. The protocol involves obtaining a force as a function of plate separation. The plate separation is then increased to the initial plate separation, and a reverse force as a function of plate separation is obtained. The two plots are typically different, indicating the stent underwent irreversible compression, and the difference may be referred to as the hysteresis.

Radial compression involves applying a uniform inwardly directed radial force. The crimp head resembles a multiple fingered chuck, which applies force uniformly around the stent circumference. As in the parallel plate compression, the chuck motion is reversed, and the force is measured as a function of radius. In both tests, the range of the test depends on the size of the stent and is generally run until a discontinuity results, i.e., buckling in the stent. Then subsequent measurements are taken just before the discontinuity results.

When internal pressure is applied to an artery, stresses develop in the longitudinal and circumferential directions. Longitudinal stress is a result of the internal pressure acting on the ends of the artery, causing strain along the axis. The circumferential (hoop) stress is the result of the radial action of the internal pressure acting on the walls of the artery, increasing its diameter. Stent deployment in an artery generates internal pressure in the artery. The effect of a crimping tool on a stent generates external pressure on the stent. The stent applies an outwardly directed radial force (RF) on the artery as it expands, which in turn generates a hoop stress that is associated with an expansive hoop force (HF) on the artery wall. Similarly, the RF imposed by the crimping tool generates a hoop stress on the stent from which a HF can be derived.

The terms chronic outward force (COF) and radial resistive force (RRF) describe the specific characteristics of the stent material and geometry. COF refers to the opening force of the stent acting on the artery as the stent tries to go back to its nominal diameter. COF is modeled as a circumferential force, and it generates hoop stress. RRF refers to the force generated by the stent to resist compression. RRF is modeled as a compressive force acting on the stent. The compression is directed circumferentially and acts as a hoop force, despite the fact that it is referred to as a radial force.

In idealized models, RF and HF may be related through an equation. In practice, RF and HF may differ in magnitude and direction and may not be directly compared. However, in tests described here, when a force cannot be measured directly, the standard thin-walled tube model will be used.

Accordingly, in in vitro tests, direct measurement of RF is performed by applied radial forces, which are directed inward in the case of crimping and outward in the case of stent expansion. The Mylar film test compresses the stent circumferentially and provides a direct measurement of the HF. This force could be expressed in terms of RRF when reporting values of stent compression and COF with values of stent expansion.

The crush test does not provide a measurement of RF or HF since the applied load is vertical. On the other hand, it is easier to measure than hoop loads and this loading mode is important in situations where stents are exposed to external crushing, such as the carotid artery.

Referring to FIG. 1A, a half-circumference of a stent 100 is shown, and a section of the stent cylinder 102 is shown. When stent 100 is internally pressurized via expansive radial force 104, circumferential stress 106 may develop. The strain in the cylinder wall 108 in turn generates a compressive radial force 110 and a hoop force 112. When a stent 100 is deployed in an artery, the expansive radial force 104 of the stent is balanced by the compressive radial force 110.

In the present disclosure, design considerations may be taken into consideration regarding how radial forces and hoop forces are distributed, since the Wenzel-Cassie microstructure on the outer surface of the stent can be tailored to alter the usual relationships between radial and hoop forces. For example, in some stent applications radial force is better tolerated than hoop force, while the converse may be true in other stent applications. In most cases, an overall reduction of hoop and radial force is desirable.

The clinical aims of stent patency and stent localization are not directly related or are decoupled from the forces when stent localization by microstructures of the present disclosure are employed. Hence, one or more embodiments of the present disclosure may provide microstructured stents which do not rely on these forces to achieve the clinical goals.

Referring to FIG. 1B, a microstructured surface 150 is described comprising a substrate 152, first microstructure 154, second microstructure 156, and third microstructure 158. The first microstructure 154 may be a two-dimensional sinusoidal microstructure. The second microstructure 156 may be a pillar microstructure. The third microstructure 158 may be a fluted microstructure. The different microstructures are arranged hierarchically. In certain embodiments, the dimensions of diameter, pitch, and height may be defined as follows: the first microstructure 154 dimensions are 1.1 to 10 times the dimensions of second microstructure 156 dimensions, the second microstructure 156 dimensions are 1.1 to 10 times the dimensions of third microstructure 158 dimensions, and the pillars may have any elliptical or polygonal cross section.

### BRIEF DESCRIPTION OF THE SEVERAL VIEWS OF THE DRAWINGS

FIG. 1A is an illustration of hoop force and radial force within a lumen.

FIG. 1B is an illustrative embodiment with a hierarchical microstructure comprising three levels.

FIG. 2 is an illustration of a relation between hoop forces and radial forces.

FIG. 3 is an illustrative embodiment of a stent with a microstructure surface.

FIG. 4 is an illustrative embodiment of a tri-part stent with a microstructure surface.

FIG. 5 is an illustrative embodiment of a stent where the microstructures conform to the contraction/expansion of the stent.

FIG. 6 is an illustrative embodiment of a microstructured stent where the radius may be adjusted in situ to minimize radial force.

FIG. 7 is an illustrative embodiment of a microstructured stent where the microstructure is localized on the stent and designed to be aligned with a particular geometrical feature of the target lumen.

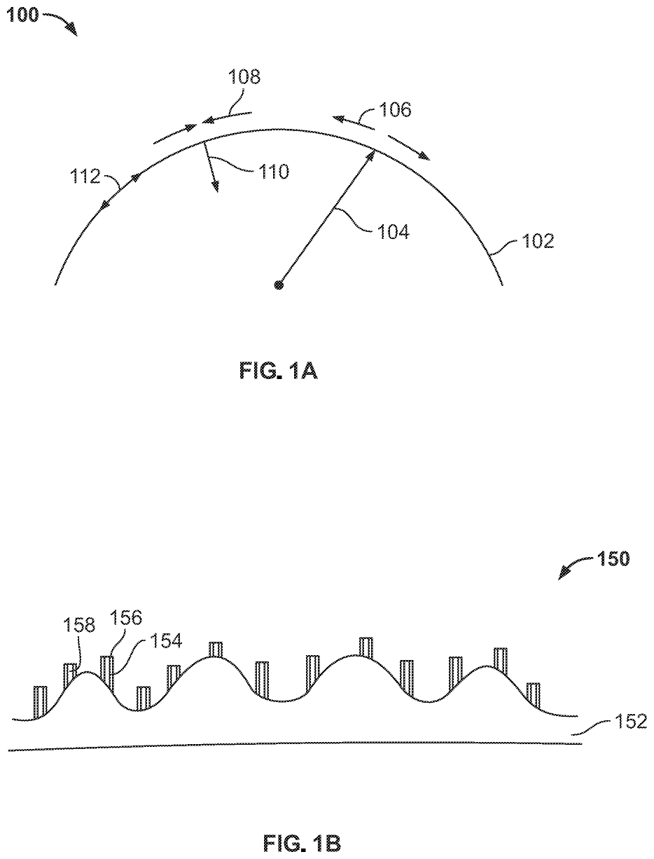

### DETAILED DESCRIPTION OF THE DISCLOSURE

Generally, the term “emergent phenomenon” as used herein will be understood to describe the interfacial structures formed between the microstructured surface of the stent and the lumen of the target tissue. The decoupling of the compression load requirements created by the target lumen from the radial force requirements may be linked to reducing stent migration in some embodiments. Accordingly, the design-to-target methodology employed in some embodiments of the present disclosure may balance axial pull tests which measure migration against radial force tests which support stent patency.

Generally, the term “composite hierarchical microstructure” as used herein will be understood to describe a hierarchical structure where each hierarchy may be a level comprised of a composite microstructure. A composite microstructure may be understood as geometrically different microstructures disposed on a single layer or substrate, or disposed in a hierarchical fashion on said substrate. Some composite hierarchical microstructures may be self-similar, i.e., a composite microstructure of one layer may be a scaled-up or scaled-down version of the microstructure on one or more other layers.

As stated, all terms, methodologies, and test circumstances are described in detail and serve as definitions for terms that appear in the disclosure. For example, shear force, hoop force, radial compressive force, radial expansive force, and hysteresis take their meaning from the protocols and test set-ups described herein.

In the evaluation of endoluminal stents, force is registered on a load cell placed at the end of a film loop placed around the stent and may be measured as a function of vertical displacement. The clinically relevant forces are the chronic outward force (COF) and the radial resistive force (RRF) for certain embodiments. The COF is the unloading force measured at nominal diameter minus 1 mm. The RRF is the force required when crimping a stent embodiment to its nominal diameter, minus 2 mm.

In the evaluation of endoluminal stent embodiments as described herein, a crush test may be employed which measures force registered on a force gauge connected to a plate that applies vertical compression. The clinically relevant force may be radial stiffness, which is radial force per unit length as a function of displacement.

In the evaluation of endoluminal stent embodiments disclosed herein, a U-shaped measuring system measures the force applied by the stent to a tubular holder linked to an electronic balance. The clinically relevant force may be radial force (RF), usually recorded as a function of the stent diameter.

In particular, in the evaluation of biliary stent embodiments disclosed herein, the radial force may be measured with a force gauge deployed inside a contracting cylinder. The clinically relevant force may be radial force, usually recorded as RF vs. diameter, and RF at 4 mm diameter.

In the evaluation of gianturco stent embodiments disclosed herein, the radial force may be measured using the mylar method. The plot obtained may be given as radial force vs. cross sectional area reduction.

In the evaluation of esophageal stent embodiments disclosed herein, radial force may be by transverse compression, circumferential compression, and/or a three-point bending test. The clinically important parameter may be the COF, measured as RF vs. diameter.

One aspect of this disclosure, and embodiments described herein, is coupling Wenzel-Cassie microstructured surfaces with various stent applications/designs and to assess the relevant clinical parameters, where oversizing may be replaced with Wenzel-Cassie bonding. This approach may yield stents with reduced radial force and reduced chronic complications. By decoupling the force requirements of the stent from the localization requirements of the stent, stent designs that are more easily delivered through minimally invasive means can be produced which are less prone to complications of traditional stent designs of the prior art.

Traditional frictional force used in localization of a traditional stent of the prior art requires large outwardly directed radial stent forces which generate a mechanical coupling between the stent and endolumenal surface. It is understood that the frictional force is proportional to the RF. Thus, for a traditional stent of the prior art, if the RF is zero, so is the frictional localization force. This is not the case for the Wenzel-Cassie non-frictional localization where the localization force is nonzero and the outwardly directed radial force can be zero.

A hierarchical microstructured surface may be understood to include a surface microstructure comprising high surface energy regions juxtaposed with lower surface energy regions. These high and low surface energy surfaces need not be stacked, but rather interlocking or in juxtaposition. In some embodiments, higher Wenzel-Cassie localization forces may be achieved when the microstructures span a three-dimensional space and are stacked.

High surface energy microstructures may be understood to be wetting. In the case of water wetting, high surface energy microstructures may be understood to be hydrophilic in some embodiments. Low surface energy microstructures may be understood to be non-wetting. In the case of water wetting, low surface energy microstructures may be understood to be hydrophobic in some embodiments. The combination of wetting and non-wetting zones in juxtaposition creates what is known as a Wenzel-Cassie interface, as disclosed herein.

It will be appreciated that a Wenzel-Cassie interface, as disclosed herein, may not require an outward radial force to create a localizing force in comparison to traditional prior art devices. In order to translate the stent relative to the endolumenal surface, energy may be applied to disrupt the low energy state of the Wenzel-Cassie interface. In some embodiments, the hierarchical microstructured surface may have reduced contact (e.g., surface area, pressure, friction, etc.) with the endolumenal surface when compared with traditional prior art devices. In certain embodiments, the stents disclosed herein may have instances of no contact at all with the endolumenal surface, but yet still creates a localizing force to fixate the stent at the target site.

The Wenzel-Cassie interface may couple the stent to the endolumenal surface via Van der Waal interactions, as one example among others, which do not necessarily involve mechanical interaction. In some embodiments, the stents of the present disclosure may include some frictional aspect, but at least a portion of the localizing force may be supplied by a non-frictional Wenzel-Cassie bond, and thus RF may be reduced and decoupled from the localization force in stents of the present disclosure relative to traditional stents of the prior art.

A Wenzel-Cassie interface may be characterized by a variety of nomenclatures in literature. A Wenzel-Cassie interface may be present if there is a “suck down” effect between the microstructured surface of a device and a target surface. A suck down effect may create a non-destructive inwardly directed RF, as opposed to a destructive outwardly directed RF. The suck down effect may reduce the energy of the Wenzel-Cassie interface creating a “bonded” condition that requires energy input to be disrupted.

For this reason, the phenomenon of Wenzel-Cassie bonding has also been called “fluid thinning.” Here, the term “Wenzel-Cassie interface” may be understood as a phenomenon that generates a fluid thinning condition in the interface between the hierarchical microstructured surface and the target surface. For example, in some embodiments the hierarchical microstructure may create capillary effects which, while technically not Wenzel-Cassie, still use fluid interaction with varying surface energy configurations and gradients to generate an inward directed RF. Thus, the hierarchical microstructured surfaces of the present disclosure may be characterized as surfaces where the area of contact between the microstructures and the endolumenal surface is reduced. In some embodiments, the area of contact may preferably be reduced by at least 25%, by at least 30%, by at least 35%, by at least 40%, by at least 45%, and most preferably by at least 50% or more. In some embodiments, the area of contact may be reduced by 75% or more. And yet in further embodiments, the area of contact may be reduced by 90% or more. And yet further still, the area of contact may be reduced by 95% or more, by 98% or more, and by 99% or more.

The microstructured surfaces of the present disclosure may, in some embodiments, be periodic. This periodicity can be leveraged to create second-order Wenzel-Cassie interfaces, which here are considered as Wenzel-Cassie interfaces. A second-order Wenzel-Cassie interface may be characterized as some type of mechanical deformation of a target surface and/or microstructured surface which creates an interlocking condition between a microstructured surface and a target surface. It will be appreciated that due to the Wenzel-Cassie interface, the interlocking of the surfaces and the deformation may be caused by mostly non-frictional forces.

For example, in some embodiments, a target surface may possess eigenwrinkle modes of deformation which causes wrinkling of the target surface without mechanical damage occurring to the target surface. One or more periodicities of the microstructured surfaces may be matched to the eigenwrinkle modes of the target surface such that the target surface and microstructure surface interlock with minimal mechanical contact. In some alternative embodiments, the microstructured surface may also possess eigenwrinkle modes, which can achieve the same second-order Wenzel-Cassie effect. In essence, shear forces may be translated into wrinkling of either the stent surface or endolumenal surface without displacement of the stent. This second-order Wenzel-Cassie effect may be particularly useful in lumens that have a peristaltic motion, such as the esophagus.

In certain embodiments of the present disclosure, a related, but different second-order Wenzel-Cassie effect may be associated with Schallamach waves. These waves may be generated in either the microstructure surface or the target surface, and comprise multiple wrinkle components, some of which may not be eigenmodes. The periodicity of the microstructured surface may “catch” some components of the Schallamach waves and allow other components to “pass by.” This phenomenon may be useful in localizing stents where there is an inhomogeneous phase passing through the lumen of a stented region, i.e., food or fecal matter. A second-order Wenzel-Cassie interface of this type may be understood to be a “slip-grip” interface as used herein.

In some embodiments, other second- and third-order Wenzel-Cassie interfaces may be incorporated, where the Wenzel-Cassie interface and the periodicity of the microstructured surface work in cooperation to localize a stent placed in a dynamically changing lumen. The periodicity of the microstructure may be further coupled cooperatively with other mechanical properties of the microstructure surface, for example, the Young's modulus of the material comprising the microstructured surface. Additionally, the geometry of the microstructured surface and how it is attached to the stent can also enhance or diminish these second-order Wenzel-Cassie effects.

Embodiments of certain stents of the present disclosure may be characterized by mechanical properties that are unlike any stents previously described in the prior art. A microstructured surface of the present disclosure employing hydrophobic and hydrophilic regions which yields stents with displacement forces that are increased when the target contact surface/lumen becomes wet, or when oil is applied, or when a surfactant is applied, has not been disclosed in the prior art. It should be appreciated that the mode of functionality and the resulting effects, as disclosed herein, is the opposite of stents populated with mechanical friction surface texture, where the in vivo environment tends to be lubricious and therefore is not conducive to, or may prevent, frictional resistance to migration.

For frictional treatments, including barbs, raised struts, surface cylinders, pyramids and the like, large radial forces must be supplied in order for the interpenetration between tissue and surface frictional geometry to be effective. Conversely, in the present disclosure, the Wenzel-Cassie interface created by a microstructure surface when wet may prevent migration through a variety of non-contact effects broadly called Van Der Waals forces. These localization forces may be understood as largely electronic and quantum mechanical rather than mechanical and classical.

In some embodiments of the current disclosure, the Wenzel-Cassie effect may create a “suck down” effect between the lumen wall and stent. Thus, rather than outward radial forces applied to the lumen for fixation, inward radial forces may be applied. This embodiment may be particularly useful in the case of aortic aneurysm stenting, where strong outward radial forces typical of the prior art devices can exacerbate the aneurysm.

It should be understood by those skilled in the art that the effects disclosed in this application should not exclude combinations of Wenzel-Cassie effects and mechanical frictional effects. In some embodiments, depending on the location and physiological requirements, one effect, Wenzel-Cassie or mechanical frictional, may be preferred over the other on discrete portions of the same stent.

In addition, further embodiments may include adhesive and lubricious surface textures. Such surface textures may be used alone or may be used in combination. Fluid thinning, and fluid thickening, are common effects due to the disordering or ordering of water molecules caused by the effect the microstructured surface's spatially distributed surface energy patterns may have on the electric dipoles in the interfacial fluid. In particular embodiments, regions of high surface energy can be juxtaposed with regions of low surface energy which may cause peristaltic waves to pass through the stent without displacing it from its target position.

By way of enabling the disclosure for one skilled in the art and providing definitions for the force terms used herein to describe the various embodiments of this disclosure, a brief description of the methods employed follows. A combination of in vitro and ex-vivo setups are useful in characterizing the various embodiments of this disclosure. All tests will be performed on laser-cut nitinol stents to illustrate the methodology, but it will be understood that various stent material(s) or stent geometry (ies) could be used in the present disclosure that satisfies the parameter constraints of the disclosure.

### Experimental Protocol 1

### Stent Crushing

A stent section was placed between two parallel plates attached to an instron machine. Two crossing points on the stent were anchored to the bottom plate to avoid rotation. During compression, only the top plate was moved. The surface area of contact between the top plate and stent was measured as a function of plate displacement. Various contact surfaces (plate) were studied, including ex vivo tissue. Stents were compressed to a clearance between plates of 5 mm. The displacement rate was 1 cm/min. Plots of plate separation vs force are of primary interest.

### Experimental Protocol 2

### Stent Crimping and Expansion

A modified four finger milling chuck was outfitted on each finger with a curved aluminum piece such that in the closed position a circular cross section was obtained, in between the curved aluminum pieces was a stretch elastomeric membrane to prevent stent “extrusion” between fingers. A small pressure transducer was embedded on each finger inner surface, such that a smooth surface was formed, on which various test surfaces could be placed.

After crimping, the chuck was actuated in the opposite direction to allow self-expansion. The stent was crimped to 15 mm in vitro. In addition, to copy crimping into a delivery system of 18 F diameter, the stent was further compressed to a diameter of 6 mm. All measurements were performed at approximately body temperature (37° C.).

In order to understand the relationship between axial and circumferential symmetry boundary conditions and to study the contact force between struts, forces were measured in a horizontal plane and in a plane tilted 9 degrees from the horizontal. The RF of a two-strut stent is determined by summing the radial reaction force (RFsum) of all the nodes. To obtain the total RF of the stent, the results of the two-strut model stent were multiplied by 80, which reflects axial and circumferential symmetry in typical stent designs.

The HF of the isolated stent portion was determined by summing the circumferential forces (HFsum1 and HFsum2) of the nodes of the lateral free surface of the stent. In some calculations, the contribution of the contact force (CF) was included in the HF. To obtain the total HF of the stent, the results of the two-strut model were multiplied by 2 (axial symmetry). RF and HF vs diameter curves were obtained.

### Experimental Protocol 3

### Stent Ex-Vivo Lumen Interaction

In all tests ovine pulmonary arteries were used. Symmetry boundary conditions were applied in the circumferential direction. Pressure was measured between stent and lumen of the artery. The extremes of the artery were fixed in the longitudinal direction to achieve inflation without length variation. In some instances, a balloon was used to supply a uniform pressure between stent and artery. To simulate stent deployment, the contact between stent and lumen was minimal on insertion, and then subsequently enabled by release of the crimping mechanism.

To study degrees of oversizing, the diameter of the ex vivo arteries were selected. Sizing was performed by applying 2.2 kPa pressure to the lumen, and the system allowed to equilibrate to measure arterial size or diameter.

The HF of the artery was determined by summing the circumferential reaction force along all the nodes of the tangential free surface (HFsum) and multiplying by 2. The increase in HF due to stenting was calculated by subtracting the HF of the artery from the HF value after pressurization.

In stent deployment, the vessel wall has a tensile HF with a characteristic direction which typically differs from the HF direction of the stent. Thus, measurement was delayed until these two directions became one in the equilibrium deployed state. Note, after stent-artery contact the HF of the artery increases and the HF of the stent decreases. The intersection of the curves determined the equilibrium point.

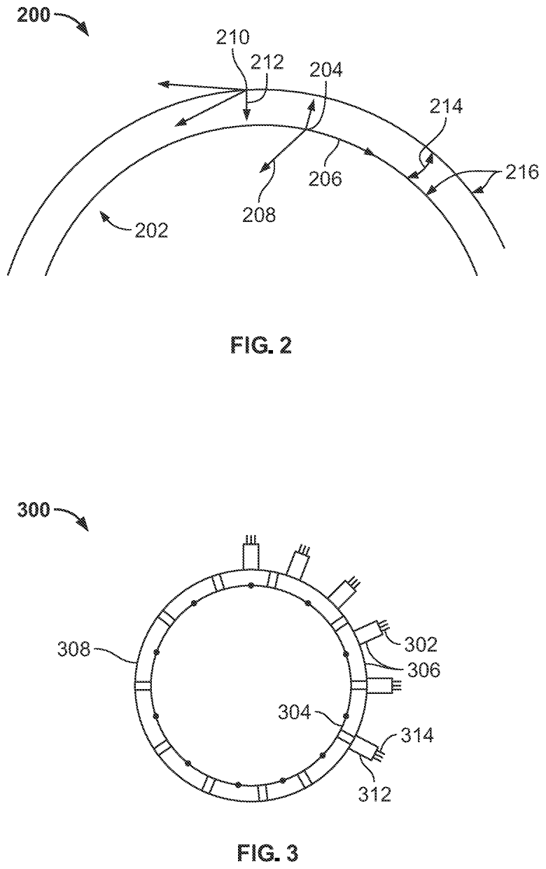

Referring now to FIG. 2, a half-circumference of a stent 200 is shown, and a section of the stent cylinder 202 is shown which illustrates the radial force RFsum. Stent strut element 204 illustrates hoop forces HFsum1 206 and HFsum2 208. Lumen element 210 illustrates hoop force HFsum 212, where Total HF=HF lumen=2HFsum. The contact force CF 214 is generated by the contact planes 216, where Total HF=HF stent=2 (HFsum1+HFsum2−CF).

The presently disclosed subject matter may be further described and illustrated by the following specific but non-limiting examples and experimental results. The following examples and experimental results may include compilations of data that are representative of data gathered at various times during the course of development and experimentation related to the presently disclosed subject matter.

### Experiment 1

Radial Size Reduction without Luminal Contact

For the following experiment, the RF and HF were determined for stents coated with a smooth coating and for stents coated with a compound pillar microstructure. All tested stents included a cylindrical geometry with no terminal flares. Referring now to FIG. 3, a stent 300 is illustrated. Stent 300 may include a lumen contacting surface 302, an internal surface 304, and a microstructure layer 306. In some embodiments, the stent wall may include nitinol struts 308. The microstructure layer 306 may include microstructures of compound pillars comprising first hierarchical layer 312 and second hierarchical layer 314.

The RF and HF values for different stent diameters are illustrated below in Table 1. Each stent included a nitinol coating with texture hysteresis curves (no contact with artery).

TABLE 1
Radial and Hoop Hysteresis of Standard Stent

| | (radial, hoop) force compression | | (radial, hoop) force decompression | |
|---|---|---|---|---|
| Stent diameter | Force Comp. | Force Comp. | Force Decomp. | Force Decomp. |
| Stent Dia. | (Radial) | (Hoop) | (radial) | (radial) |
| 32 mm | 0 (N) | 0 (N) | 0 | 0 |
| 30 mm | 4.2 | 0.2 | 4.2 | 0.2 |
| 28 mm | 21 | 1.5 | 15 | 1.4 |
| 26 mm | 25 | 3.5 | 17 | 2.1 |
| 24 mm | 29 | 4.1 | 18 | 2.5 |
| 22 mm | 33 | 5.1 | 20 | 3.5 |
| 20 mm | 35 | 6.0 | 21 | 4.1 |
| 18 mm | 36 | 6.1 | 30 | 4.8 |
| 15 mm | 38 | 6.3 | 38 | 6.3 |

It should be noted that the nitinol coating thickness was adjusted so there was no detectable difference in the hysteresis curves for coated smooth and coated microstructure. Table 1 is represented by smooth and microstructured stents.

A characteristic of traditional prior art stents is that the forces during stent delivery are higher than after stent delivery at the same diameter. This hysteresis is one of the primary reasons why smooth stents must be oversized in order that enough post-delivery radial force remains to fixate the stent in the lumen. Note the radial force is much more strongly hysteretic than the hoop force, which means increasing the radial force has little effect on stent patency. Consequently, there is no reason for a large radial force except to overcome hysteresis and provide an adequate frictional localizing force. The hysteresis may be beneficial if patency is decoupled from providing localizing frictional force.

Note also, in esophageal stenting, patient comfort is a function of stent length and radial force, not hoop force. In esophageal stenting, discomfort becomes the primary adverse event of stenting when the final lumen diameter is greater than the minimum diameter necessary to achieve lumen patency. In traditional stenting applications, unnecessarily increasing the lumen diameter beyond the diameter clinically needed can result in lumen deterioration. For example, in aortic aneurysm stenting, stenting can cause more dilation of the artery. In coronary stenting, the sudden change in lumen diameter creates thrombogenic turbulence.

### Experiment 2

Radial Force Reduction with Luminal Contact

The hysteresis problem of traditional prior art stents can be described as follows: if one wishes to increase post-delivery RF, one must substantially increase delivery RF, which is a limiting factor in developing the stent design. To meet these demands, the design will likely result in increasing the minimal delivery diameter of the stent. Thus, there is a need to reduce the post-delivery RF specification in order to make stent delivery practical in a wider variety of applications.

To test the effect of microstructured surface on the requirements of post-delivery RF regarding slippage or migration of the stent, it should be recognized one cannot simply pull on the stent relative to the lumen. Most stents are designed to reduce their radius when an axial force is applied. Consequently, direct application of an axial stress will narrow the stent in a way that does not reflect the phenomenon of migration in vivo.

Axial forces are not substantial in vivo, and this fact is one reason why a delivery strategy which involves stent diameter reduction by applying an axial force may provide a more practical application. Nevertheless, there are localized axial displacements of the stent versus the lumen which add up over time. The magnitude of these small displacements can be quantified by fixing the stent diameter and measuring the shear force for a particular diameter. To do this, an incompressible cylinder must be introduced in the lumen of the stent to prevent the stent from changing diameter under axial stress. This is a realistic measure of a stent's properties because axial displacement is a sum of these small non-radius changing displacements, and not a macroscopic, large axial force effect which would cause the stent to narrow.

The experimental set-up includes using an artificial lumen, obtainable from manufacturers such as Syndaver, which is fitted to the target post-deployment of the stent. Various post-deployment RF can be obtained precisely by varying the diameter of the artificial lumen. The cylindrical form that prevents stent narrowing under axial force must fit the post-delivery radius of the stent and not add any radial or hoop force, and further should be incompressible. A variety of PVC cylinders in increments of 0.5 mm diameter in the expected range of the post-delivery stent diameter may be used.

As shown in TABLE 2, the addition of a microstructured surface reduces the need for a high post-delivery RF for microstructured stent surfaces. TABLE 2 compares smooth and microstructured surfaces vs axial shear (slippage) for various post-delivery RF.

TABLE 2
Microstructured Vs Smooth Stent Shear Force

| Post Delivery RF* | Smooth Stent | Microstructured Stent |
|---|---|---|
| 0.5 (N) | 1.5 g/cm² | 112 g/cm² |
| 1.3 | 3.5 | 182 |
| 4.1 | 9.9 | 218 |
| 9.3 | 21 | 239 |

*The post-delivery RF is given in total force (Newtons)rather than pressure to be consistent with TABLE 1.

The effect of RF on stent design may be determined by axial and/or circumferential stent symmetry. In some embodiments, an increase in RF may be achieved by adding struts. The effect of HF, on the other hand, may depend only on the axial symmetry in some embodiments. Note, for individual strut elements, the RF may be considerably lower than the HF. However, in embodiments having the cylindrical symmetry, this relation inverts, and the RF may far exceed the HF. This result may be interpreted as the tangential HF vectors picking up radial components of force when the strut elements are joined in a cylindrical geometry.

It should also be noted that in certain embodiments, end-flare geometries are not considered. In the regions where an end-flare geometry is employed, the RF may be substantially increased, since the flare induces a radial moment caused by bending in the axial direction in addition to the HF conversion to RF caused by the cylindrical geometry. Thus, for flared stent designs, the ability to reduce delivery RF may be constrained, with minimal reduction in delivery RF achieved by reducing strut number and strut diameter. Thus, in some embodiments, there may be a need to eliminate a flared stent geometry. The traditional rationale for a flared stent geometry is the reduction of post-delivery stent migration.

It is also noted that in some embodiments, the greater the compression (high RF), the greater the hysteresis. In these embodiments, increasing the RF by adding struts or increasing the diameter of the struts may result in diminishing returns in post-delivery RF relative to delivery RF, exacerbating the delivery RF problem.

Surprisingly, in some embodiments, is has been found that stents with the highest ratio of post-delivery RF to delivery RF are those stents with the lowest post-delivery RF. This relation makes the present disclosure, as characterized in TABLE 2, particularly useful. Thus, microstructured stents may be lighter weight, easier to deliver, and can present less foreign surface when the post-delivery RF requirements are reduced. Post-delivery RF requirements may largely be driven by migration issues. Whereas, maintaining an open lumen (the purpose of stents) may be improved with higher HF. The present disclosure provides embodiments disclosing stents that may convert RF to HF while increasing shear force (decreasing migration).

### Experiment 3

### Crimping or Delivery Diameter

To assess the effects of crimping diameter on RF and HF, the hysteresis for embodiments having different radial displacements was studied. The RF on the crimping tool and HF in the stent correspond to a change of slope in the loading curve below some threshold radius (in these studies 8 mm dia.). Beyond the radial thresholds, RF may increase dramatically from 8 mm down to 18 F (6 mm dia.). The emergence of this generic nonlinear relationship in stents between delivery RF and crimp diameter may cause the delivery RF to increase with relatively no increase in post-delivery RF in some embodiments.

For some embodiments, the source of the nonlinearity may involve self-contact between struts. Changing the usual Hooke's law relationship between force and displacement may become overwhelmed by a volumetric relationship which involves powers of the displacement. In other words, the reaction force in response to compression may become overwhelmed by a contact force, without increasing the reaction force.

The larger the percentage difference (as a percentage of relaxed stent radius) of some embodiments between the radial displacement of the stent when in contact with the lumen and the radial displacement at delivery, the greater the decoupling of delivery RF from post-delivery RF. Surprisingly, across a wide variety of stent types, the percentage difference may lie in a relatively narrow range, and hence may be a limiting characteristic in some embodiments of the disclosed stents herein.

Below, in TABLE 3, the typical change in hysteresis as a function of delivery diameter is provided.

TABLE 3
Delivery Diameter Vs Hysteresis

| Delivery | Radial Stent Force (g/cm²) | | Hoop Stent Force (g/cm²) | |
|---|---|---|---|---|
| Dia. (mm) | Pre-delivery | Post-delivery | Pre-delivery | Post-delivery |
| 30 | 1.2 | 1.2 | 0.3 | 0.3 |
| 28 | 8.7 | 8.7 | 1.1 | 1.1 |
| 26 | 19 | 17 | 2.2 | 1.9 |
| 24 | 23 | 20 | 4.2 | 3.1 |
| 20 | 35 | 21 | 5.5 | 3.3 |
| 15 | 38 | 22 | 6.0 | 3.5 |
| 10 | 40 | 25 | 6.1 | 4.2 |
| 6 | 48 | 48 | 7.8 | 7.8 |

### Experiment 4

### Stent-Lumen Interaction

In Experiment 4, the RF and HF were studied to compare smooth coated stents and microstructured coated stents. The microstructured stent is of the type disclosed in Experiment 1.

In this set of experiments, the studies aim to compare HF versus the diameter of the stent after a maximum crimping force (CF) of 7.3 N. The deployment force (DF) and the equilibrium force (EF) were recorded. It should be noted that the stiffness of the artery may affect the equilibrium force and equilibrium diameter.

For smooth coated stents, the stent surface may contact the arterial lumen. As the lumen expands, the stent surface may lose contact with the arterial lumen surface and therefore, the lumen surface and stent surface may be relatively friction free. This may result in small shear forces.

TABLE 3
30 mm Stent Deployment in Ovine Aorta (CF =
7.3, DF = 2.2, EF = 0.4)

| Aorta Diameter | Hoop Force (smooth) | Hoop Force (microstructure) |
|---|---|---|
| Aorta Dia. | Hoop Force (Smooth) | Hoop Force (Microstructure) |
| 30 | 0 (N) | 0 (N) |
| 28 | 1.1 | 1.7 |
| 26 | 2.1 | 3.8 |
| 24 | 2.4 | 4.0 |
| 22 | 3.0 | 5.1 |
| 20 | 3.1 | 5.5 |

### Experiment 5

### Oversized Stent-Artery Interaction

To evaluate the effect of oversized stents on lumenal integrity, an ovine artery was used. In TABLES 4-7 below, the hoop stress of ovine pressurized arteries (10 kPa) before and after stenting is given for different degrees of stent oversizing.

TABLE 4
Hoop stress of ovine pressurized arteries (10 kPa) before
and after stenting Vs stent oversizing (smooth).

| 10 kPa = 1.5 psi | 1 psi = 52 mmHg | Max Hoop Stress | |
|---|---|---|---|
| Pressurized diameter | Stent Over Size | (above baseline 10 kPa) | |
| 27 mm | 10% | 25 | kPa |
| 25 mm | 20% | 72 | kPA |
| 23 mm | 30% | 86 | kPa |

TABLE 5
Hoop stress of ovine pressurized (10 kPa) arteries before
and after stenting Vs stent oversizing (microstructured).

| 10 kPa = 1.5 psi | 1 psi = 52 mmHg | Max Hoop Stress | |
|---|---|---|---|
| Pressurized diameter | Stent Over Size | (above baseline 10 kPa) | |
| 27 mm | 10% | 37 | kPa |
| 25 mm | 20% | 102 | kPA |
| 23 mm | 30% | 115 | kPa |

TABLE 6
HF and RF at deployment and equilibrium
for different oversizes (Smooth).

| Over size % | Deployment HF/RF (N) | Equilibrium HF/RF (N) |
|---|---|---|
| 10 | 1.9/13.4 | 0.4/3.1 |
| 20 | 2.7/15.1 | 0.9/5.3 |
| 30 | 2.9/17.3 | 1.5/8.5 |
| 40 | 3.0/18.1 | 1.7/11.7 |

TABLE 7
HF and RF at deployment and equilibrium for
different oversizes (microstructured).

| Over size % | Deployment HF/RF (N) | Equilibrium HF/RF (N) |
|---|---|---|
| 10 | 3.5/9.9 | 3.7/0.2 |
| 20 | 3.7/11.5 | 3.8/0.3 |
| 30 | 4.3/12.5 | 3.9/0.5 |
| 40 | 5.5/14.1 | 4.7/2.3 |

The values of HF and RF are recorded at deployment and at equilibrium for different sizes of oversizing. At 10% oversizing for some embodiments, the maximum physiological stress is exceeded only on the lumenal side, and the hoop stress of the stented artery doubles the value of the physiologic condition. At 20% oversizing (RF=16N) for some embodiments, the maximum physiological stress is triplicated on the lumenal side and across most of the artery wall. Oversizing values higher than 30% (RF=17.5N) for some embodiments, greatly exceeded the maximum physiological stress along the entire stented area, reaching the outer surface of the artery.

TABLE 8
Maximum Radial Stress (smooth stent)
in Ovine Aorta (N = 50 sections)

| | |
|---|---|
| Failure Pressure Range | % Failures (long time) |

| | | |
|---|---|---|
| <50 | kPa | 12 |
| 50-70 | kPa | 46 |
| 70-110 | kPa | 26 |
| >110 | kPa | 16 |

TABLE 9
Microstructure Supported Hoop Stress
in Ovine Aorta (N = 50 sections)

| | |
|---|---|
| Failure Pressure Range | % Failures (long time) |

| | | |
|---|---|---|
| <200 | kPa | 2 |
| 200-250 | kPa | 18 |
| 250-300 | kPa | 62 |
| >300 | kPa | 18 |

Failure is defined as a circumference change greater than 1 mm/min for two minutes after the equilibrium time (>1 minute after deployment).

### Experiment 6

### Bend Test/Bend Flexibility

Bend flexibility is defined by a three-point bend test where the stent is constrained at ends 6 times the stent diameter apart. The flexibility is the force required to displace the center of the stent by the same distance as the stent's diameter.

TABLE 10
Bend Force (N).

| | Commercially | Low Radial |
|---|---|---|
| | Available | Force Stents |
| Acceptable Force Range | 135-90N | 105-55N |

In some embodiments, stent comfort may depend on minimizing the tendency of a stent to straighten the lumen anatomy.

### Experiment 7

### Peristaltic Motion Flexibility

The following experimental set-up consists of a soft-bodied swallowing robot having a conduit diameter of 20 mm and a length of 200 mm made up of ECOFLEX 00-30. The robot has been developed to mimic the human swallowing process under different rheological and medical conditions. The robot can generate peristaltic waves of different characteristics similar to the waves generated when food bolus travels from the upper to the lower esophagus in humans. A successful transit of the bolus is referred to as an effective peristalsis. An ineffective peristalsis is often associated with the residue of food particles left in the esophagus.

The robot consists of twelve equally spaced layers, and in each layer, four pneumatic chambers are present surrounding the conduit of the robot. The purpose of these chambers is to deform sequentially layer by layer under the pressure of supplied air in such a way that the shape of the deformation can take the form of a traveling peristaltic wave from the top layer to the bottom layer. The swallowing robot (SR) is a realistic example of the biological process of swallowing. Performing tests on such a robot can widen the range of different measurement and evaluation schemes as compared to human studies.

A stent migration experiment was performed utilizing the SR to determine the displacement of a stent from its initial position to a displaced position due to the effect of the travel of the peristaltic contractile waves of different characteristics through the layers of the robot, generated by the pneumatic valves.

Two different stent dimensions were used, one of 23 mm×28 mm×100 mm and a second of 18 mm×21 mm×150 mm. The displacement data is the average of the two different stent dimensions used in the experiments. The experiments were conducted at approximately room temperature (36° C.). The conduit of the robot and the surface of the stent were kept wet by applying a dry mouth spray before performing each experiment. A baseline pressure of 25 psi was maintained in all the chambers throughout the experiments.

A speed of 20 mm/s was used to simulate the peristaltic wave. The trajectory of the waves used to simulate peristalsis were inspired by pressures typically found in humans during the same process. The profile of the waves was kept the same for all experimental runs, and the amplitude was scaled by factors of 1, 1.2, 1.5, and 2. By changing the amplitude, one varies the pressure in the actuation chambers and hence, the displacement of the 10 mm wide adjacent chamber heads. By varying the scaling factor, different radial compressive forces of the conduit occlusion were achieved and assessed relative to the migration under the influence of peristaltic motion.

TABLE 11
Stent Migration as Function of Peristaltic
Cycles at Scaling Factor 1.2

| No. Cycles | Displacement (mm) |
|---|---|
| 1 | 0.00 |
| 2 | 0.72 |
| 3 | 2.53 |
| 4 | 3.42 |
| 7 | 3.66 |
| 21 | 3.69 |
| 34 | 4.13 |

### Experiment 9

### Shear Migration Force

In some embodiments, a stent may reduce in radius when an axial force is applied while in contact with a lumen. The reduction in radius can significantly reduce the radial force, in some cases the radial force may become close to zero. Thus, in a shear test where the stent is translated axially with respect to a lumen, the stent must be constrained from reducing radius while not applying additional radial force. For stents that supply less than 1 kPa the stent should be oversized (lumen reduced by sutures) to exert 1 kPa outward radial pressure. Frequently, this last step is not done, and not performing this step in such testing should be considered flawed testing.

The experimental setup was as follows, various stents were deployed and measured in an artificial lumen or one retrieved from an animal. The lumen was fixed by at least 4 points on one end to one of the (tensile tests) Instron heads. The inner volume of the stent was filled with an incompressible lightweight mandrel. The distal stent end was connected to the moving head of the Instron in the same way the lumen was attached to the other head of the Instron.

The stent was deployed at a target radial force or oversized radius with respect to the relaxed inner radius of the lumen. Artificial lumens are typically not elastic but have the lubricious characteristics of most biological lumen. The elastic aspect of natural lumens is usually not important since axial displacement sufficient to elongate the lumen is a more than sufficient anti-migration force. Normally the displacement rate is 0.5 to 1 N/sec, typically 1 N/sec. The results are generally not sensitive to this rate, as long as the time to migration is greater than 1 second.

Generally, the migration force is measured as a 10% time derivative discontinuity, but generally, the release resulting in migration is sudden.

TABLE 12
Migration Force (Low Radial Force Vs No Texture)

| Stent Type | Max Force (N) |
|---|---|
| Smooth p1 | 0.5 |
| p1 (texture) | 5.4 |
| T13 | 7.8 |
| T29 | 3.2 |
| T9 | 4.3 |
| T18 | 4.3 |
| T26 | 4.1 |
| T24 | 3.4 |
| S31 | 7.5 |
| S27 | 5.7 |
| S25 | 4.2 |

### Summary of Experiments

The above experiments serve to establish safe level limits, performance limits, and localization efficacy with stents utilizing microtextured surfaces. The results demonstrate that in some embodiments having a microstructured surface, one may achieve localization of a stent without relying on radial forces to generate friction as required by traditional stents of the prior art. In the following portion of this disclosure, device specifications are disclosed that may be useful for embodiments having low radial force stents. Low radial forces may be achievable due to the active adhesive functionality of microstructured surfaces used in applications involving placing a stent inside a hollow body cavity.

The device specifications given in this section may be applicable to all stenting operations currently performed on humans, from coronary stents to esophageal stents. Consequently, the specifications will be given in percentages (relative parameters) rather than absolute units. Where an embodiment differs appreciably from the range of specifications provided herein, specifications will be given for that specific embodiment. All specifications are given in terms of ranges because tissue characteristics, stent materials, stent geometries, and stent dimensions differ. However, it has been discovered as a result of this work that many of the disclosed embodiments tend to fall within the specifications given. Furthermore, the specifications enabled by a microstructured surface stent (low radial force) may be significantly different from non-textured stents. The differences in specifications are clinically significant.

### Radial Force

Compressional radial force is a device specification that affects ease of use. Traditional stents of the prior art generally require high radial compression forces to achieve radial dimensions suitable for deployment in their intended application and therefore present several technical challenges. Deployment devices may generally be bulkier in order to reliably deliver the needed radial compression force to properly deliver the stent device to a target location. The compression profile is also important. The compression profile may be obtained by measuring the compression radial force for at least two radial reductions. Typically, the stent radius may be reduced by about 10% to 20% in some embodiments. However, too much radial compression may risk permanently deforming the stent and rendering it less effective or ineffective for some embodiments.

### Stent Specification 1, Compression Profile:

In some embodiments, assuming the radial pressure at ΔR=0 is 0 Newtons (N), then if the radial pressure at ΔR=10% is X then the radial force at ΔR=20% may be in the range 1.10× to 1.5×, preferably 1.10× to 1.25×.

It will be understood that in the art radial compression may often be reported in units of force. Using units of force may require certain assumptions about the dimensions of the stent. The conversion from force in Newton to Pascals is 1 g/cm²=100 Pa, and area is calculated from A=dlπ, where “d” is the diameter of the stent and “1” is the length of contact between stent and body lumen.

Some contemporary stent compression profiles are 2.0 and more. This may be allowed if ΔR=10% generates a pressure less than 1.8 kPa. But then a compression profile of 2.0 means the ΔR=20% is unnecessarily high. This does not affect the patient but may place a higher demand on the deployment device design. It also significantly increases stent hysteresis, which is associated with a cascade of design and patient adverse effects. The ideal stent should have a compression profile of about 1.

TABLE 13
Based on capability of deployment devices

| ΔR = 10% | ΔR = 20% | Safe Compression Profile |
|---|---|---|
| 1.8 kPa | 2.7 kPa | 1.50 |

| | |
|---|---|
| Traditional Stents | 2.50-1.40 |
| Low Radial Force Stents | 1.25-1.10 |

### Stent Specification 2, Maximum Radial Compression Force:

In some embodiments, the maximum radial compression force for low radial force stents may be between 2.7 kPa and 1.5 kPa, preferably between 2.26 kPa and 1.50 kPa, more preferably between 2.0 and 1.5 kPa.

### Stent Specification 3, Radial Expansion Profile:

In some embodiments, assuming the radial pressure at ΔR=0 is 0 Newtons (N), if the radial pressure at ΔR=10% is X then the radial force at ΔR=20% is in the range 1.09× to 1.42×, preferably 0.91× to 1.18×, more preferably 0.91× to 1.10×.

For certain embodiments, the stent should undergo no change in radial force between compression and expansion. As described previously, the difference is called hysteresis.

TABLE 14
Based on tissue necrosis and comfort

| ΔR = 10% | ΔR = 20% | Safe Compression Profile |
|---|---|---|
| 1.28 kPa | 1.82 kPa | 1.42 |

| | |
|---|---|
| Stents in current use | 2.10-1.27 |
| Low Radial Force Stents | 1.10-1.00 |

### Stent Specification 4, Maximum Radial Expansion:

In some embodiments, the maximum radial expansion force for low radial force stents may be between 1.82 kPa and 1.00 kPa, preferably between 1.62 kPa and 1.00 kPa, more preferably between 1.38 and 1.00 kPa.

### Axial Force

Stent Specification 5, Minimum Peristaltic Shear Force (comfort):

In some embodiments, it will be appreciated that the desired specifications herein may not be met with traditional stents of the prior art. Embodiments of the low radial force stents of the present disclosure may resist migration by finding an equilibrium position of about 20-32 psi (1.2 scale factor), preferably between 25-32 psi, more preferably 29-32 psi.

### Stent Specification 6, Minimum Peristaltic Shear Force (Max Force):

In some embodiments, it will be appreciated that the desired specifications herein may not be met with traditional stents of the prior art. Embodiments of the low radial force stents of the present disclosure may resist migration by finding an equilibrium position of about 20-35 psi, preferably 25-32 psi, more preferably 29-32 psi.

It will be understood that this test applies both axial (shear) and radial forces, in natural combination. The easily measurable force is the radial force applied to the peristaltic actuators. The shear stress may be derived from this pressure and many characteristics of the peristaltic motion, defined above. Therefore, the minimum peristaltic shear force may be given in terms of a radial pressure applied to 10 mm actuators.

TABLE 15
Minimum Peristaltic Shear Force

| | Minimum Pressure (psi) | Satisfied |
|---|---|---|
| No surface pattern | 20-25 psi | NO |
| Low radial force (max) | 20-35 psi | YES |

### Stent Specification 7, Minimum Shear Force.

Certain embodiments of stents of the present disclosure may include greater than 3.0 N shear force at a radial expansion pressure of less than 1 kPa, preferably greater than 4.75 N, and more preferably greater than 5.0 N. (Experiment 9).

### Stent Specification 8, Maximum Bend Force.

Certain embodiments of stents of the present disclosure may include a bend force between 105-55 N, preferably 96-55 N, and more preferably 67-55 N.

Bend force may also be a combination of axial and radial forces and may be a measure of flexibility. See Experiment 6 (above). In some embodiments, stent comfort may depend on minimizing the tendency of a stent to straighten lumen anatomy.

### Hoop Force

### Stent Specification 9, Maximum Hoop Stress (Compression):

In some embodiments of the present disclosure, the hoop force of compression may be less than 8.5% of the radial force in compression at stent diameter reduction of 50%, preferably less than 7.8%, and more preferably less than 6.5%. (Experiment 1)

### Stent Specification 10, Maximum Hoop Stress (Decompression):

In some embodiments of the present disclosure, the hoop force of decompression may be less than 8.1% of the radial force in expansion at stent diameter reduction of 50% after compression to 55%, preferably less than 7.3%, and more preferably less than 6.1%. (Experiment 1)

### Hysteresis

### Stent Specification 11, Maximum Hoop Hysteresis:

In some embodiments of the present disclosure, the hysteresis ([compression force−decompression force]/compression force) may be less than 35% at 20% stent radius reduction after reduction to 25%, preferably 25%, and more preferably 20%. (Experiment 1)

### Stent Specification 12, Maximum Radial Hysteresis:

In some embodiments of the present disclosure, the hysteresis ([compression force−decompression force]/compression force) may be less than 37% at 20% stent radius reduction after reduction to 25%, preferably 31%, and more preferably 25%. (Experiment 1)

### Stent Specification 13, Post-Delivery Hysteresis Shear Force:

In comparing certain embodiments of a non-textured stent and a textured stent of the present disclosure to each other (same stent design, other than texture), the textured stent may have a shear force of at least 10 times the shear force of the non-textured design, preferably at least 15 times, more preferably at least 20 times for stents compressed and then deployed (Experiment 2)

### Stent Specification 14, Post-Delivery Hysteresis Shear Force:

In comparing certain embodiments of a non-textured stent and a textured stent of the present disclosure to each other (same stent design, other than texture), the textured stent may have a shear force at least 20 times the shear force of the non-textured stent when deployed at a post-delivery radial force of 2 kPa. (Experiment 2)

### Stent Specification 15, Post-Delivery Hysteresis Shear Force:

In comparing certain embodiments of a non-textured stent and a textured stent of the present disclosure to each other (same stent design, other than texture), the textured stent should have a shear force at least 10 times the shear force of the non-textured stent when deployed at a post-delivery radial force of 20 kPa. (Experiment 2)

### Stent Specification 16, Post-Delivery Hysteresis Shear Force:

In comparing certain embodiments of a non-textured stent and a textured stent of the present disclosure to each other (same stent design, other than texture), the textured stent may have a higher relative shear force ratio (shear force present disclosure/shear force non-textured) at 2 kPa compared to at 20 kPa. (Experiment 2)

### Stent Specification 17, Pre-Post Hysteresis:

In some embodiments having a stent of the present disclosure, the stent may undergo a hysteresis of less than 5% when compressed 50% and returned to the pre-compressed stent size, preferably less than 3%, and more preferably less than 1%.

It should be understood that a useful baseline for hysteresis is to measure the compressional force from the starting diameter to the minimum diameter (metal on metal) and back to the original diameter, and the change in the compressional force (pre force) from the expansion force (post force). In traditional stents of the prior art, the post force will frequently be less than pre force, which poses a serious problem when the stent size is matched to the lumen size. Usually, clinicians assume some hysteresis and adjust the stent size accordingly. Therefore, there is a need for a stent that returns to its designed size with minimal hysteresis loss thereby allowing for more accurate matching of the stent to the lumen size. This advantage of some embodiments of the stents disclosed herein may occur when the ends of the stent are kept open by a ring of textured coating. Unlike metal struts, plastic coating may not undergo permanent deformation in the same way that metal does. Furthermore, the textured coating may supply radial force of its own by being radially attractive. When one combines these effects, the results of experiment 3 may be obtained.

TABLE 16
Pre-Post Hysteresis

| | |
|---|---|
| Radial Stent Force | Hoop Stent Force |

| | | | | |
|---|---|---|---|---|
| Delivery Dia. | Pre | Post | Pre | Post |

| | | | | | |
|---|---|---|---|---|---|
| 30 | mm | 1.2 | 1.2 | 0.3 | 0.3 |
| 28 | mm | 8.7 | 8.7 | 1.1 | 1.1 |
| 26 | mm | 19 | 17 | 2.2 | 1.9 |
| 24 | mm | 23 | 20 | 4.2 | 3.1 |
| 20 | mm | 35 | 21 | 5.5 | 3.3 |
| 15 | mm | 38 | 22 | 6.0 | 3.5 |
| 10 | mm | 40 | 25 | 6.1 | 4.2 |
| 6 | mm | 48 | 48 | 7.8 | 7.8 |

The following examples and embodiments are meant to be illustrative only, and not in any way limiting.

### Example 1

Embodiment of a stent with improved contact area.

One of the main sources of chronic irritation of the esophagus due to the implantation of an esophageal stent is the flared structures at the ends of the stent, which such structure can be found in the prior art. The flares can have a radius as much as twice the main body of the stent. The flares can be a major design limitation since flared stents are more difficult to compress to their delivery size than non-flared stents. The flares on esophageal stents of the prior art are provided only to increase the radial force of the stent and attempt to reduce stent migration. The embodiments having a low radial force stents of the present disclosure increase shear force without increasing radial force and discomfort. Higher shear stress may reduce the area of the stent that needs to make contact with the esophagus.

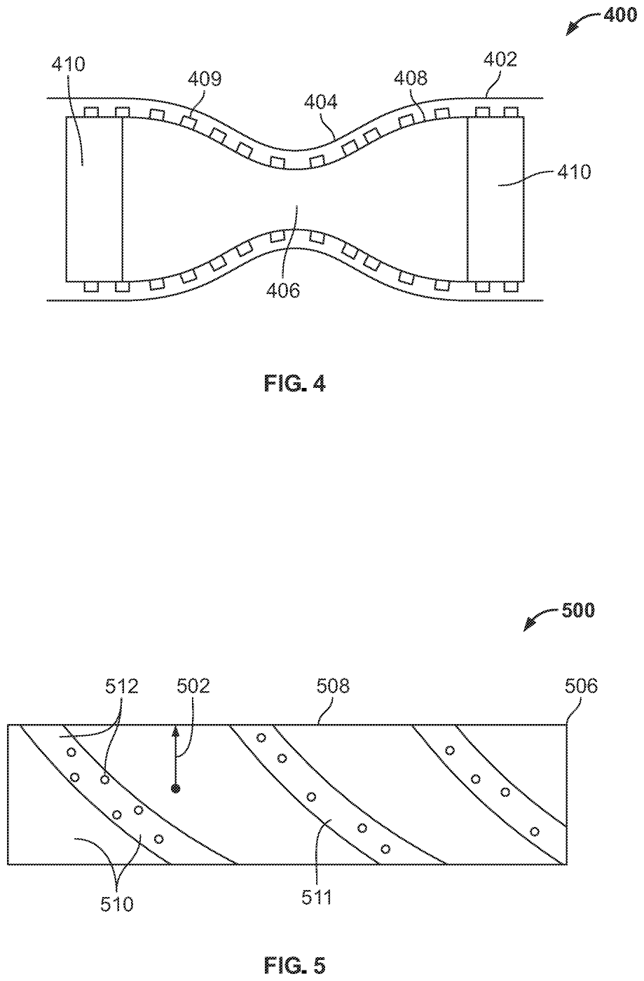

Referring to FIG. 4, a stent 400 for keeping patent a lumen 402 with a stricture 404 comprises a stricture expanding element 406, an elastomeric lumen 408 and two anchoring rings 410. The elastomeric lumen 408 has microstructure 409 of the present disclosure in a ring around each end of the soft elastomeric lumen 408. The stricture expanding element 406 is comprised of a wire mesh which has short axial length but sufficient radial force to expand the stricture 404. The elastomeric lumen 408 is a continuously smooth surface tube comprised of silicone or similar material. The anchoring rings 410 are slightly oblique so that they can be compressed on the major axis. As pictured in FIG. 4, the stricture expanding element 406 is first placed in the region of the stricture 404, then the elastomeric lumen 408 is placed such that the expanding elements is located midway the axial length of the soft elastomeric lumen 408. The two ends of the elastomeric lumen 408 are secured with the anchoring rings 410, by compressing each along their major axis to a smaller width and securing the lumen ends.

### Example 2

### An Embodiment of a Low Compression Profile Stent

Referring to FIG. 5, a low compression profile stent is a stent 500 with a maximum radius 502 less than 50% of the radius of a standard stent 504 of the same intended implant radius. The radius of the ends 506 is the same radius as the mid body 508. The stent is coated in an elastomer 510, with a spiral ribbon of microstructure 511.

### Example 3

An Embodiment of a Stent with Low Radial Expansion Profile

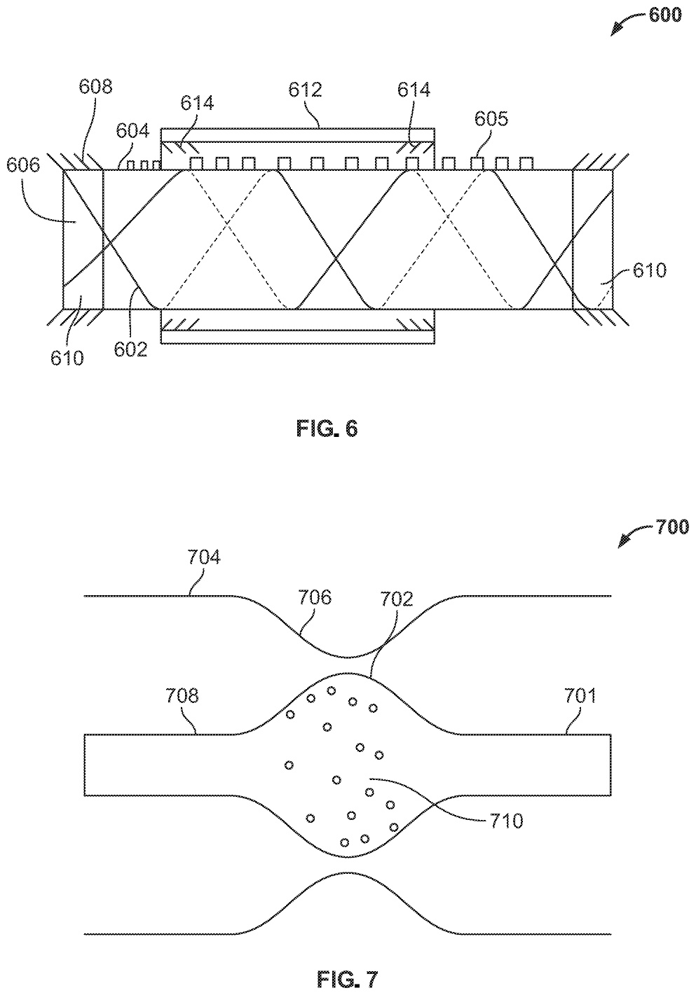

Referring to FIG. 6, a low radial expansion force profile stent 600 comprises a helix 602 coated with a soft polymer 604 with a microstructure texture 605. The helix 602 is normally elongated with a radius smaller than the intended implant radius. The helix 602 has bands 606 with a ratchet texture 608 pointing distally. The band 606 is attached to the helix 602 distally at ends 610. The band 606 passes through a hub 612 with ratchet in the proximal direction. When the stent 600 is implanted, the band 606 is pulled proximally and the ratchet texture 608 on the band 606 engage with ratchets in the hub 614, shortening the stent 600 axial length. The helix 602 expands and makes a snug fit to the target lumen. The band 606 is made of a durable plastic. At the time of explanation, the stent can be relaxed by cutting the band 606. The radial expansion profile is in the range of 1.09× and 1.42×.

### Example 4

An Embodiment of a Stent with a Reduce Contact Area

Referring to FIG. 7, A metal wire woven stent 700 with a bulge 702 in the middle is to be implanted in a lumen 704 with a stricture 706. The bulge 702 is to be placed proximal to the stricture 706. The stent is coated with a soft polymer 708. In the bulge 702 region of the coating there is disposed a microstructure texture 710. The stent 700 has a maximum shear force greater than 3.0 N at a radial expansion of less than 1 kPa.

What is claimed is:

1. An implantable device for placement in a body lumen comprising:
a tubular member having an outer surface and an inner surface, the outer surface comprising a substrate, the substrate further comprising at least one first hierarchical microstructure pattern and at least one second hierarchical microstructure pattern, wherein the at least one first hierarchical microstructure pattern and the at least one second hierarchical microstructure pattern are arranged to generate a Wenzel-Cassie state.

2. The implantable device of claim 1 wherein the at least one first hierarchical microstructure pattern and at least one second hierarchical microstructure pattern comprises a two-dimensional sinusoidal microstructure, a pillar microstructure, or a fluted microstructure.

3. The implantable device of claim 1, the substrate further comprises at least one third hierarchical microstructure pattern.

4. The implantable device of claim 3, wherein the at least one third hierarchical microstructure pattern is a fluted microstructure.

5. The implantable device of claim 1, wherein the at least one first hierarchical microstructure pattern comprises dimensions which are 1.1 to 10 times the dimensions of the at least one second hierarchical microstructure pattern.

6. The implantable device of claim 3, wherein the at least one second hierarchical microstructure pattern comprises dimensions which are 1.1 to 10 times the dimensions of the at least one third hierarchical microstructure pattern.

7. The implantable device of claim 2, wherein the pillar microstructure comprises an elliptical or polygonal cross section.

8. The implantable device of claim 1, wherein the tubular member comprises a maximum radial compressive pressure in the range of 1.82 kPa to 1.00 kPa.

9. The implantable device of claim 1, wherein the substrate comprises an adhesive surface texture.

10. The implantable device of claim 1, wherein the substrate comprises an lubricious surface texture.

11. The implantable device of claim 1, wherein the substrate comprises nitinol struts.

12. The implantable device of claim 11, wherein the tubular member comprises a nitinol coating with texture hysteresis curves.

13. The implantable device of claim 1, wherein the tubular member further comprises a stricture expanding element, a soft elastomeric lumen, and at least one anchoring rings.

14. The implantable device of claim 13, wherein the stricture expanding element comprises a wire mesh.

15. The implantable device of claim 1, wherein the tubular member comprises an elastomer coating.

16. The implantable device of claim 15, wherein at least one microstructure pattern is disposed of in a spiral-ribbon pattern.

17. The implantable device of claim 1, wherein the tubular member comprises a metal woven wire, the metal woven wire further comprises a bulge.

18. The implantable device of claim 17, wherein the tubular member comprises a soft polymer coating.

19. The implantable device of claim 18, wherein the at least one first hierarchical microstructure pattern and/or the at least one second hierarchical microstructure pattern are disposed upon the soft polymer coating.

20. An implantable device for placement in a body lumen comprising:
a tubular member having an outer surface and an inner surface, the outer surface comprising a substrate, the substrate further comprising at least one first hierarchical microstructure pattern, at least one second hierarchical microstructure pattern, and at least one third hierarchical microstructure pattern, wherein the at least one first hierarchical microstructure pattern, the at least one second hierarchical microstructure pattern, and the at least one third hierarchical microstructure pattern are arranged to generate a Wenzel-Cassie state.
